(19) United States
(12) Patent Application Publication
(10) Pub. No.: US 2026/0060919 A1
(43) Pub. Date: Mar. 5, 2026

(54) TOPICAL ANALGESIC FOR RAPID RELIEF OF JOINT AND MUSCLE PAIN WITHOUT NUMBING AGENTS

(71) Applicant: Manooj Persad, New York, NY (US)

(72) Inventor: Manooj Persad, New York, NY (US)

(21) Appl. No.: 19/269,183

(22) Filed: Jul. 15, 2025

Related U.S. Application Data
(60) Provisional application No. 63/688,082, filed on Aug. 28, 2024.

(51) Int. Cl. A61K 9/00; A61K 9/06; A61K 31/60; A61K 36/9066; A61K 47/10; A61K 47/20

(52) U.S. Cl. CPC A61K 9/0014; A61K 9/06; A61K 31/60; A61K 36/9066; A61K 47/10; A61K 47/20

(57) ABSTRACT

A topical analgesic formulation designed for rapid pain relief in joints and muscles comprises a skin penetration agent, specifically dimethyl sulfoxide (DMSO), in a concentration range of 30% to 60% by mass, and curcumin in a concentration range of 3% to 6% by weight. The formulation is characterized by the absence of anesthetic agents and the ability to provide pain relief within one hour of application. Additional components may include methyl salicylate in a concentration range of 5% to 10% by weight and menthol in a concentration range of 2% to 4% by weight. The formulation is presented in a gel form and includes instructions for application. This composition is particularly suitable for topical application to alleviate discomfort in affected areas.

### CROSS-REFERENCE TO RELATED APPLICATION

This application claims the benefit of and priority from U.S. Provisional Patent Application Ser. No. 63/688,082, filed Aug. 28, 2024, and entitled, “TOPICAL ANALGESIC FOR RAPID RELIEF OF JOINT AND MUSCLE PAIN WITHOUT NUMBING AGENTS.” The disclosure of the prior application is considered part of and hereby incorporated by reference in its entirety in the disclosure of this application.

### BACKGROUND

Management of localized discomfort, particularly in muscles and joints, is a significant concern in healthcare and wellness. Current options for addressing such discomfort often involve compounds that provide temporary relief. While these options are widely available, they may not meet the needs of all users. For instance, individuals engaged in activities that demand precise sensory feedback may find the common numbing effects of certain compounds to be a hindrance, as these effects can interfere with their ability to perform tasks that require a high degree of tactile sensation.

### BRIEF DESCRIPTION OF DRAWINGS

FIG. 1 depicts the application of a topical analgesic to the skin.

FIG. 2 depicts the chemical structure of a compound used in the formulation of one embodiment of a topical analgesic.

FIG. 3 depicts the chemical structure of a compound used in the formulation of one embodiment of a topical analgesic.

### DETAILED DESCRIPTION

The present disclosure proposed in this patent includes a method to achieve rapid relief of joint and muscle pain by combining the anti-inflammatory ingredients found in Oil of Wintergreen and Curcumin with skin penetration enhancers DMSO and ethoxydiglycol. This method achieves rapid relief by rapidly penetrating the outer layer of skin (Stratum Corneum) and delivering inflammation reducing medicine targeted to only the affected muscles and joints. By rapidly targeting the inflammation that causes pain this method avoids the use of anesthetics present in standard rapid relief joint and muscle pain relievers. Standard rapid relief joint and muscle topical pain relievers such as Aspercreme®, Absorbine® Jr (Pro) and Salonpas® provide relief through the use of the anesthetic lidocaine which numbs the nerves in the affected area. The problem with the numbing effect on nerves is that it hinders the tactile and sensory response that many people rely on to effectively and safely perform their jobs. For example, people who handle power tools or heavy equipment can have disastrous results if they lack proper sensory feedback from their hands, fingers and feet. Numbness can also affect the performance of other groups such as athletes, artists, musicians, professional gamers etc. This invention addresses these problems by relieving the inflammation that causes pain instead of masking the pain by numbing it.

The formulation disclosed herein provides a solution to the aforementioned issues by offering a topical analgesic that does not rely on numbing agents to achieve pain relief. Instead, the formulation focuses on rapid skin penetration to deliver anti-inflammatory agents directly to the inflamed tissues, thereby addressing the root cause of pain. The formulation is designed to be fast-acting, with the intent to begin alleviating pain within an hour of application, which aligns with industry standards for fast-acting products. By avoiding the use of anesthetic agents, the formulation allows users to maintain their tactile abilities while benefiting from effective pain management.

In the following description, specific details are set forth, but embodiments of the technologies described herein may be practiced without these specific details. Well-known circuits, structures, and techniques have not been shown in detail to avoid obscuring an understanding of this description. Phrases such as “an embodiment,” “various embodiments,” “some embodiments,” and the like may include features, structures, or characteristics, but not every embodiment necessarily includes the particular features, structures, or characteristics.

Some embodiments may have some, all, or none of the features described for other embodiments. “First,” “second,” “third,” and the like describe a common object and indicate different instances of like objects being referred to. Such adjectives do not imply objects so described must be in a given sequence, either temporally or spatially, in ranking, or any other manner. The terms “comprising,” “including,” “having,” and the like, as used with respect to embodiments of the present disclosure, are synonymous. Terms modified by the word “about” include amounts that vary slightly from the meaning of the unmodified term, such as varying by a relative +/−5%. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

Reference is now made to the drawings, which are not necessarily drawn to scale, wherein similar or same numbers may be used to designate the same or similar parts in different figures. The use of similar or same numbers in different figures does not mean all figures including similar or same numbers constitute a single or same embodiment. Like numerals having different letter suffixes may represent different instances of similar components. The drawings illustrate generally, by way of example, but not by way of limitation, various embodiments discussed in the present document.

In the following description, for purposes of explanation, numerous specific details are set forth in order to provide a thorough understanding thereof. It may be evident, however, that the novel embodiments can be practiced without these specific details. In other instances, well-known structures and devices are shown in block diagram form in order to facilitate a description thereof. The intention is to cover all modifications, equivalents, and alternatives within the scope of the claims.

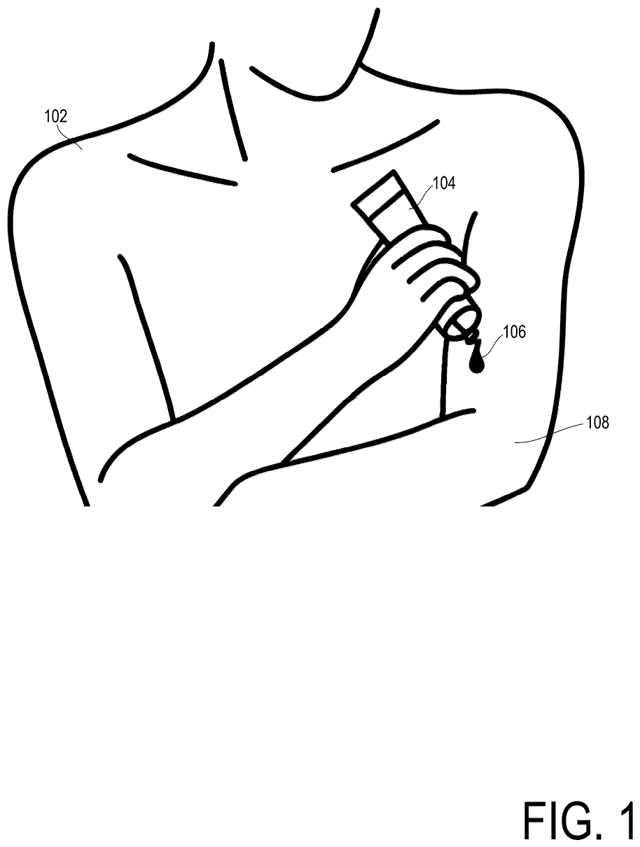

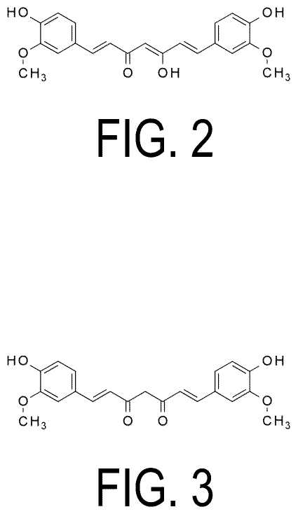

In an illustrative embodiment, a formulation of a liniment includes dimethyl sulfoxide (DMSO), methyl salicylate, menthol, curcumin, ethanol, as well as various inactive ingredients, such as stabilizers, emulsifiers, etc. FIG. 1 shows one embodiment of a user 102 applying the liniment 106 from a container 104 to the user's elbow 1081. Unless stated otherwise, all formulations are given by weight. In one embodiment, the formulation includes 50% DMSO, 10% methyl salicylate, 3% menthol, 3% curcumin, and 20% ethanol, with the remainder various stabilizers. In another embodiment, the formulation includes 50% DMSO, 10% methyl salicylate, 3% menthol, 1% curcumin, 29.5% isopropyl alcohol, 5.5% caprylic/capric triglyceride, and 1% ethoxdiglycol. More generally, in some embodiments, the formulation may include 30%-60% DMSO, 2%-30% methyl salicylate, 1%-10% menthol, 0.5%-10% curcumin, 15%-54% ethanol, and 5%-30% various stabilizers and/or other inactive ingredients. In other embodiments, the formulation may include 50%-55.5% DMSO, 7%-15% methyl salicylate, 1%-3% menthol, 25%-50% isopropyl alcohol, 1%-3% curcumin, 1%-10% caprylic/capric triglyceride, and 1%-10% ethoxdiglycol. The curcumin may be either the enol form (as shown in FIG. 2) or the keto form (as shown in FIG. 3). In another embodiment, the formulation includes 50% methylsulfonylmethane, 10% methyl salicylate, 4% camphor, 5.5% olive squalane, 1% ethoxdiglycol, 3% arnica, and 26% isopropyl alcohol. In another embodiment, the formulation includes 50% methylsulfonylmethane, 10% methyl salicylate, 4% camphor, 5.5% olive squalane, 1% ethoxdiglycol, 3% arnica, and 26% isopropyl alcohol. In other embodiments, the formulation may include 50%-55.5% methylsulfonylmethane, 7%-15% methyl salicylate, 4%-11% camphor, 1%-10% olive squalane, 1%-10% ethoxdiglycol, 1%-3% arnica, and 25%-50% isopropyl alcohol. The formulations described above may provide fast-acting pain relief and/or anti-inflammation.

The formulation may include any suitable type and/or amount of inactive ingredients, such as antioxidants, preservatives, chelating agents, pH adjusters, solubilizers, emulsifiers, humectants, etc. For example, the formulation may include as antioxidants 0.01%-0.1% butylated hydroxytoluene (BHT) and/or butylated hydroxyanisole (BHA), 0.01%-1% tocopherol (vitamin E), and/or 0.01%-0.5% ascorbic acid (vitamin C). The formulation may include as preservatives 0.01%-0.3% parabens (e.g., methylparaben, propylparaben) and/or 0.5%-2% benzyl alcohol. The formulation may include as chelating agents 0.01%-0.1% ethylenediaminetetraacetic acid (EDTA) and/or 0.1%-0.5% citric acid. The formulation may include as pH adjustors suitable amounts of sodium hydroxide, hydrochloric acid, and/or citric acid to adjust the pH to, e.g., 4-10. The formulation may include as solubilizers and/or emulsifiers 0.1%-2% polysorbates (e.g., tween 20 and/or tween 80) and/or 0.1%-2% lecithin. The formulation may include as humectants 1%-10% glycerin and/or 1%-10% propylene glycol.

The formulation has several potential use cases and benefits. It is particularly beneficial for individuals suffering from joint and muscle pain, including athletes, craftsmen, artists, and individuals engaged in activities that require precise sensory feedback. Unlike numbing agents commonly found in other topical analgesics, this formulation allows users to retain their tactile abilities while experiencing effective pain management. It should further be appreciated that the formulations disclosed herein are fact-acting. As used herein, a fact-acting analgesic formulation is one which provides pain relief within 15 minutes or less. In some embodiments, the formulation may take longer, such as 15-60 minutes, to take effect.

The specific percentages and ranges of these ingredients have been carefully determined through experimentation and testing. The formulation ranges disclosed herein correspond to the ranges that provide optimal function.

In small-scale, non-randomized, non-placebo-controlled trials, participants reported fact-acting, effective relief of joint and muscle pain. The formulation used in the trials, includes 50% DMSO, 10% methyl salicylate, 3% menthol, 1% curcumin, 29.5% isopropyl alcohol, 5.5% caprylic/capric triglyceride, and 1% ethoxdiglycol. Data was collected from a study involving a group of 80 people of varying ages, professions, and gender. Each participant was provided with a sample of the formulation. Each participant was asked to try the sample for a period of 30 days and then fill out a short questionnaire listing their, age, gender, profession, and average time to recover after the sample was applied. The data from those questionnaires were collated into the tables below.

TABLE 1

| | GENDER | | | |
|---|---|---|---|---|
| Experience | Male | Female | Unspecified | TOTAL |
| Pain relief within 1 hour | 42 | 28 | 2 | 72 |
| Pain Relief within 2 hours | 4 | 2 | 0 | 6 |
| Pain relief within 3 hours | 1 | 1 | 0 | 2 |
| No relief | | | | 0 |
| TOTAL | | | | 80 |

TABLE 2

| | AGE CATEGORIES | | | | | |
|---|---|---|---|---|---|---|
| Experience | Under 15 | 15-25 | 25-50 | 50-67 | Senior | TOTAL |
| | | | | | | 0 |
| Pain relief within 1 | 5 | 10 | 24 | 10 | 23 | 72 |
| hour | | | | | | |
| Pain Relief within 2 | 0 | 1 | 1 | 2 | 2 | 6 |
| hours | | | | | | |
| Pain relief within 3 | 0 | 0 | 1 | | 1 | 2 |
| hours | | | | | | |
| No relief | | | | | | 0 |
| TOTAL | | | | | | 80 |

TABLE 3

| | CAUSE OF PAIN | | | | | |
|---|---|---|---|---|---|---|
| | | | | Work related - | | |
| | | | | handling tools and | | |
| | | Hobby - | | machinery, typing, | | |
| | Athletics, | Videogaming, | | lifting, driving, | Other- | |
| | Sports, | Painting, | Health | standing for | could not | |
| | Exercise, | Guitar and | related - | long periods | determine | |
| Experience | Workouts | Drum playing | Arthritis | of time | cause | TOTAL |
| | | | | | | 0 |
| Pain relief within 1 | 16 | 7 | 16 | 32 | 1 | 72 |
| hour | | | | | | |
| Pain Relief within 2 | 0 | 1 | 2 | 1 | 2 | 6 |
| hours | | | | | | |
| Pain relief within 3 | 0 | 0 | 1 | | 1 | 2 |
| hours | | | | | | |
| No relief | | | | | | 0 |
| TOTAL | | | | | | 80 |

The specific percentages and ranges of these ingredients have been carefully determined through experimentation and testing. The formulation ranges disclosed herein correspond to the ranges that provide optimal function.

In an illustrative embodiment, a user applies the formulation directly to the skin at an area where pain relief is required, such as fingers, hands, wrists, elbows, shoulders, and/or the like. The user's hand functions as the applicator for the topical analgesic formulation. The user dispenses a predetermined amount of the formulation from a container onto the affected area. The user then uses the fingers to spread the formulation evenly over the skin, ensuring adequate coverage of the affected area. The spreading action facilitates the penetration of the formulation into the skin to reach the inflamed tissues. The method depicted in FIG. 1 allows for direct application of the topical analgesic formulation to the area of discomfort. This targeted approach ensures that the anti-inflammatory agents contained within the formulation are delivered efficiently to the site of inflammation. The method is designed to be simple and user-friendly, allowing for self-administration of the analgesic without the need for additional tools or assistance.

A user may apply, e.g., about 5 milliliters per application, and may apply the formulation every, e.g., 2-4 hours, up to 5 applications per day. The particular recommended amounts may depend on the specific formulation and any limitations on daily amounts of specific active ingredients in the formulation.

In an illustrative embodiment, the formulation is preferably in the form of a liniment rather than a cream, as liniments have been found to achieve better skin penetration and delivery of the active ingredients. The liniment is applied topically to the affected area, and a thin layer is sufficient for effective pain relief.

The invention offers a unique approach to pain management by targeting inflammation, which is the underlying cause of pain. By addressing inflammation rather than simply numbing the area, the formulation provides a more comprehensive and long-lasting solution. The absence of numbing agents also eliminates the undesirable side effects associated with numbing, allowing users to perform their tasks or engage in activities without any hindrance.

### EXAMPLES

Illustrative examples of the technologies disclosed herein are provided below. An embodiment of the technologies may include any one or more, and any combination of, the examples described below.

Example 1 includes an analgesic formulation comprising, by weight 30%-60% dimethyl sulfoxide (DMSO); 2%-30% methyl salicylate; 1%-10% menthol; 1%-10% curcumin; and 15%-54% ethanol.

Example 2 includes the subject matter of Example 1, and wherein the analgesic formulation does not include a numbing agent.

Example 3 includes the subject matter of any of Examples 1 and 2, and wherein the analgesic formulation does not include a steroid.

Example 4 includes the subject matter of any of Examples 1-3, and wherein the analgesic formulation is fast-acting.

Example 5 includes the subject matter of any of Examples 1-4, and including, by weight, 2-4% curcumin.

Example 6 includes the subject matter of any of Examples 1-5, and including, by weight, 2-4% menthol.

Example 7 includes the subject matter of any of Examples 1-6, and including, by weight 45-55% dimethyl sulfoxide (DMSO); 8-12% methyl salicylate; 2-4% menthol; and 2-4% curcumin.

Example 8 includes the subject matter of any of Examples 1-7, and including, by weight 50% dimethyl sulfoxide (DMSO); 10% methyl salicylate; 3% menthol; and 3% curcumin.

Example 9 includes the subject matter of any of Examples 1-8, and wherein the analgesic formulation is a liniment.

Example 10 includes a method for applying a topical analgesic formulation comprising rubbing the analgesic formulation into an area of skin of a user, wherein the analgesic formulation comprises, by weight 30%-60% dimethyl sulfoxide (DMSO); 2%-30% methyl salicylate; 1%-10% menthol; 1%-10% curcumin; and 15%-54% ethanol.

Example 11 includes the subject matter of Example 10, and wherein the analgesic formulation does not include a numbing agent.

Example 12 includes the subject matter of any of Examples 10 and 11, and wherein the analgesic formulation does not include a steroid.

Example 13 includes the subject matter of any of Examples 10-12, and wherein the analgesic formulation is fast-acting.

Example 14 includes the subject matter of any of Examples 10-13, and including, by weight, 2-4% curcumin.

Example 15 includes the subject matter of any of Examples 10-14, and including, by weight, 2-4% menthol.

Example 16 includes the subject matter of any of Examples 10-15, and including, by weight 45-55% dimethyl sulfoxide (DMSO); 8-12% methyl salicylate; 2-4% menthol; and 2-4% curcumin.

Example 17 includes the subject matter of any of Examples 10-16, and including, by weight 50% dimethyl sulfoxide (DMSO); 10% methyl salicylate; 3% menthol; and 3% curcumin.

Example 18 includes the subject matter of any of Examples 10-17, and wherein the analgesic formulation is a liniment.

Example 19 includes the subject matter of any of Examples 10-18, and wherein rubbing the analgesic formulation into an area of skin of a user comprises rubbing about 5 milliliters of the analgesic formulation into an area of skin of the user.

Example 20 includes the subject matter of any of Examples 10-19, and including reapplying the analgesic formulation an additional 1-4 times per day, as needed.

1. An analgesic formulation comprising, by weight:
30%-60% dimethyl sulfoxide (DMSO);
2%-30% methyl salicylate;
1%-10% menthol;
1%-10% curcumin; and
15%-54% ethanol.

2. The analgesic formulation of claim 1, wherein the analgesic formulation does not include a numbing agent.

3. The analgesic formulation of claim 2, wherein the analgesic formulation does not include a steroid.

4. The analgesic formulation of claim 1, wherein the analgesic formulation is fast-acting.

5. The analgesic formulation of claim 1, comprising, by weight, 2-4% curcumin.

6. The analgesic formulation of claim 5, comprising, by weight, 2-4% menthol.

7. The analgesic formulation of claim 1, comprising, by weight:
45-55% dimethyl sulfoxide (DMSO);
8-12% methyl salicylate;
2-4% menthol; and
2-4% curcumin.

8. The analgesic formulation of claim 1, comprising, by weight:
50% dimethyl sulfoxide (DMSO);
10% methyl salicylate;
3% menthol; and
3% curcumin.

9. The analgesic formulation of claim 1, wherein the analgesic formulation is a liniment.

10. A method for applying a topical analgesic formulation comprising:
rubbing the analgesic formulation into an area of skin of a user,
wherein the analgesic formulation comprises, by weight:
30%-60% dimethyl sulfoxide (DMSO);
2%-30% methyl salicylate;
1%-10% menthol;
1%-10% curcumin; and
15%-54% ethanol.

11. The method of claim 10, wherein the analgesic formulation does not include a numbing agent.

12. The method of claim 11, wherein the analgesic formulation does not include a steroid.

13. The method of claim 10, wherein the analgesic formulation is fast-acting.

14. The method of claim 10, comprising, by weight, 2-4% curcumin.

15. The method of claim 14, comprising, by weight, 2-4% menthol.

16. The method of claim 10, comprising, by weight:
45-55% dimethyl sulfoxide (DMSO);
8-12% methyl salicylate;
2-4% menthol; and
2-4% curcumin.

17. The method of claim 10, comprising, by weight:
50% dimethyl sulfoxide (DMSO);
10% methyl salicylate;
3% menthol; and
3% curcumin.

18. The method of claim 10, wherein the analgesic formulation is a liniment.

19. The method of claim 10, wherein rubbing the analgesic formulation into an area of skin of a user comprises rubbing about 5 milliliters of the analgesic formulation into an area of skin of the user.

20. The method of claim 10, further comprising reapplying the analgesic formulation an additional 1-4 times per day, as needed.
